(19) United States
(12) Patent Application Publication
(10) Pub. No.: US 2020/0188345 A1
(43) Pub. Date: Jun. 18, 2020

(54) ARTICLES OF MANUFACTURE AND METHODS OF TREATMENT FOR ANEMIA

(71) Applicant: Trevor Peroval Castor, Arlington, MA (US)

(72) Inventor: Trevor Peroval Castor, Arlington, MA (US)

(73) Assignee: Trevor P. Castor, Woburn, MA (US)

(21) Appl. No.: 16/706,754

(22) Filed: Dec. 8, 2019

Related U.S. Application Data
(60) Provisional application No. 62/777,096, filed on Dec. 8, 2018.

(51) Int. Cl. A61K 31/295; A61K 9/48; A61K 9/20; A61K 9/08; A61K 31/05; A61K 36/906

(52) U.S. Cl. CPC A61K 31/295; A61K 9/4808; A61K 36/906; A61K 9/08; A61K 31/05; A61K 9/20

(57) ABSTRACT

Embodiments of the present invention are directed to formulations and methods of treating iron deficiency anemia. The dosage form has an effective amount of an iron supplement with an effective amount of a gingerol composition to suppress nausea and/or gastric distress and to promote hematopoiesis.

### GOVERNMENT SUPPORT

Embodiments of the present invention were conceived and reduced to practice without Federal sponsorship or funding.

### FIELD OF THE INVENTION

Embodiments of the present invention are directed to articles of manufacture in the form of formulations and methods of treatment of anemia.

### BACKGROUND OF THE INVENTION

Anemia is a condition in which the blood lacks enough red blood cells or the red blood cells lack enough hemoglobin to carry an adequate amount of oxygen to the tissues of the body for normal functions. A number of medicaments have been used in the past to treat anemia associated with iron deficiency. These include iron supplements and folic acid supplements. These medicaments are associated with nausea and upset stomach.

There is a need for improved formulations which do not cause gastric distress and nausea.

### SUMMARY OF THE INVENTION

Embodiments of the present invention are directed to formulations and methods of treating iron deficiency anemia. One embodiment of the present invention is directed to a dosage form for the treatment of iron deficiency anemia. The dosage form has an effective amount of an iron supplement with an effective amount of a gingerol composition to suppress nausea and/or gastric distress. The iron supplement is selected from the group comprising ferrous sulfate, ferrous gluconate, ferrous fumarate and mixtures thereof. The gingerol composition is selected from the group of gingerol compounds comprising 6-gingerol, 8-gingerol, 10-gingerol, 12-gingerol, 6-shogaol, 8-shogaol and 10-shogaol as single compositions and mixtures thereof.

One dosage form of the present invention further comprises an oil base wherein the iron supplement is dispersed as a solid suspension in such oil base and said gingerol composition is dissolved in the oil base. The oil base with the iron supplement and gingerol composition can be administered orally as a liquid or held in a gel cap.

Other embodiments of the present invention feature tablets and capsules formulations. By way of example, without limitation, one dosage form features a gingerol composition that is used to wet a powder of the iron supplement to form a gingerol wetted iron supplement powder. The gingerol wetted iron supplement powder is pressed into a tablet or loaded into a capsule.

Another embodiment features a dosage form wherein said iron supplement and gingerol are dissolved or suspended in a solution. This solution is administered orally as a liquid.

The gingerol composition may be synthesized or is an extract from a ginger compound containing natural source. For example, without limitation, a preferred ginger compound comprises gingerols and shogoals found in an extract from Zingibier officinale. A preferred gingerol composition is present in an amount effective to promote hematopoiesis.

The iron supplement is present in 40 to 80 mg of elemental iron for adult dosage forms. A preferred iron supplement is ferrous sulfate.

A further embodiment of the present invention is directed to a method of treating anemia comprising the step of administering a dosage form comprising an effective amount of an iron supplement with an effective amount of a gingerol composition to suppress nausea and/or gastric distress. The gingerol composition is selected from the group of gingerol compounds comprising 6-gingerol, 8-gingerol, 10-gingerol, 12-gingerol, 6-shogaol, 8-shogaol and 10-shogaol as single compositions and mixtures thereof. The iron supplement is selected from the group comprising ferrous sulfate, ferrous gluconate, ferrous fumarate and mixtures thereof.

The dosage form may take many different forms including an emulsion, dispersion or solution in oil base for administration as an oral liquid or held in gel caps. The dosage form may comprise capsules and tablets.

The dosage form preferably has, for adults, 40 to 80 mg of elemental iron. For example, an amount of 325 mg of ferrous sulfate is about 65 mg of elemental iron.

A preferred gingerol composition is present in an amount effective to promote hematopoiesis. Thus, the gingerol composition addresses the nausea and gastric distress of iron supplements and synergistically promotes hematopoiesis to treat anemia.

These and other features and advantages of the present invention will be apparent to those skilled in the art upon viewing the figures and reading the detailed description that follow.

### BRIEF DESCRIPTION OF THE FIGURES

FIG. 1 depicts a dosage form embodying features of the present invention;

FIG. 2 depicts a dosage form, in cross-section, embodying features of the present invention;

FIG. 3 depicts a dosage form embodying features of the present invention; and

FIG. 4 depicts an apparatus for making gingerol compositions.

### DETAILED DESCRIPTION OF THE INVENTION

Embodiments of the present invention will now be described in detail with respect to formulations and methods of treating iron deficiency anemia as to the inventors' present best mode to practice the invention. This best mode may change over time as new considerations become known or available. Embodiments of the present invention are also subject to alterations and modifications such that the present teaching and description should not be considered limiting.

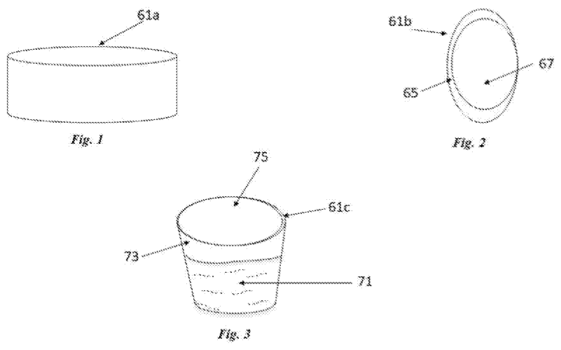

One embodiment of the present invention is directed to a dosage form for the treatment of iron deficiency anemia. Dosage forms embodying features of the present invention are, designated by the numerals 61a, 61b, and 61c, are depicted in FIGS. 1, 2 and 3. FIG. 1 depicts a tablet 61a dosage form. FIG. 2 depicts a gel cap 61b dosage form in cross section. And, FIG. 3 depicts a solution 61c dosage form. Other dosage forms, not illustrated, are by way of example without limitation, a capsule, powder for reconstitution, lozenge and the like.

Each dosage form, tablet 61a, gel cap 61b, and solution 61c, has an effective amount of an iron supplement with an effective amount of a gingerol composition to suppress nausea and/or gastric distress. The iron supplement is selected from the group comprising ferrous sulfate, ferrous gluconate, ferrous fumarate and mixtures thereof. Methods of making ferrous sulfate, ferrous gluconate and ferrous fumarate are known and the compositions can be acquired from numerous sources. The dosage form preferably has, for adults, 40 to 80 mg of elemental iron. For example, an amount of 325 mg of ferrous sulfate is about 65 mg of elemental iron.

The gingerol composition is selected from the group of gingerol compounds comprising 6-gingerol, 8-gingerol, 10-gingerol, 12-gingerol, 6-shogaol, 8-shogaol and 10-shogaol as single compositions and mixtures thereof. One preferred extract has 6-gingerol, 8-gingerol, 10-gingerol, and 6-shogaol, in which 6-shogaol and 6-gingerol define a ratio and the ratio of 6-shogaol to 6-gingerol is 0.04 to 0.40. Although the applicant does not wish to be bound to any theory, it is believed this ratio of 6-shogaol to 6-gingerol improves the efficacy of the extract for the treatment of nausea.

A further aspect of the invention is directed to an extract of ginger rhizome wherein the ginger rhizome has a starting mass and the extract has a mass associated with one or more of the following 6-gingerol, 8-gingerol, 10-gingerol, and 6-shogaol. The ratio of 6-gingerol, 8-gingerol, 10-gingerol, and 6-shogaol total mass to starting mass is 20-400/%.

A further aspect of the invention is directed to an extract having 15-25% 6-gingerol, 1-5% 8-gingerol, 1-5% 10-gingerol and 1-5% 6-shogaol.

Each dosage form, such as tablet 61a, gel cap 61b, and solution 61c, has a dosage of a gingerol composition and in one aspect the dosage is in a range of 20-40 mg of the extract of ginger rhizome. This amount of extract preferably has 4.00-14 mg of combined gingerols and shogaol.

An effective amount of iron supplement and an effective amount of gingerol composition can be divided into two or more dosage forms to address specific needs for greater amounts of iron supplement. For example, without limitation, a person with greater needs for iron supplements may take two dosage forms, such as tablet 61a, gel cap 61b and solution 61c. The amount of gingerol composition is preferably an amount to address the normal nausea and gastric distress associated with iron supplement therapy. Gingerol compositions may also be present in an effective amount to promote hematopoiesis.

Turning now to FIG. 1 and dosage form 61a, as depicted the dosage form comprises a tablet of circular shape. However, those skilled in the art will recognize that tablets may be formed in many shapes. Tablet 61a has an iron supplement, such as ferrous sulfate in effective amount to treat anemia. One example of an effective amount is 325 mg ferrous sulfate. This quantity is wetted with an effective amount of an extract having 4.00 to 14 mg of gingerol composition. The gingerol composition may also serve to protect the ferrous sulfate from oxidation. The ferrous sulfate may be combined with normal and customary excipients, diluents, binders and lubricants for tablets such as talc, lactose, starch, stearic acid and the like to facilitate adsorption of the gingerol composition and provide a compressible bulk formulation mass which can be compressed into a tablet. The bulk formulation may also be packed into capsules [not shown] in a manner known in the art.

Turning now to FIG. 2 and dosage form 61b, as depicted the dosage form comprises a gel cap having an ovoid shape. However, those skilled in the art will recognize that gel caps may be formed in other shapes as well. The gel cap comprises an outer gel surface 65 and an inner liquid 67. The outer surface 65 is known in the art and need not be further described here. The inner liquid comprises a suspension of an effective amount of an iron supplement to treat anemia. One example of an effective amount is 325 mg ferrous sulfate. This quantity is suspended in an effective amount of an extract having 4.00 to 14 mg of gingerol composition held in an oil base. The gingerol composition and oil base may also serve to protect the ferrous sulfate from oxidation.

One embodiment features an oil with an antioxidant, that is, the antioxidant is dissolved in or suspended in the oil. One antioxidant is tocopherol. A preferred formulation has an oil having one or more emulsifying agents. The emulsifying agents facilitate bioavailability and maintain the other components of the formulation in the oil base. A preferred emulsifying agent is selected from one or more of the following agents: lecithin, and short chain, medium chain and long chain triglycerides. A preferred oil is olive oil.

Processes and methods for loading the oil with the suspended iron supplement and dissolved gingerol composition are known in the art.

Turning next to FIG. 3 and dosage form 61c, a solution designated as numeral 71 is depicted in the cut-away. The solution is held in an individual administration cup 73 sealed with a removable lid 75. Those skilled in the art will recognize that solution 71 can be contained in other containers for dispensing and administration including by way of example with limitation, droppers, large bottles with or without administration cups, pouches and the like. The solution comprises a base such as water in which the effective amount of gingerol composition is held in an emulsion with ferrous sulfate particles as a suspension. The user is instructed to shake or mix the contents well.

Each dosage form is used to treat anemia, such as iron deficiency anemia, by orally taking the dosage form, such as tablet 61a, gel cap 61b and solution 61c, to provide iron. Iron supplements are commonly ingested one to three times daily. The gingerol compound prevents nausea and gastric distress and promotes hematopoiesis. Wetting the iron supplement with the oil base and/or the gingerol composition delays absorption leading to less gastric distress and prevents oxidation of the iron composition.

One preferred gingerol composition is formed from a dried powdered biomass of ginger rhizome. This dried biomass is placed in a vessel with carbon dioxide under super critical, near critical or critical conditions to form a saturated biomass powder. The carbon dioxide is separated from the biomass to form a carbon dioxide fluid extract containing the composition of gingerols and shogaol.

Preferably, the carbon dioxide is held at a temperature of 20-50 degrees Celsius (° C.), at a pressure of 1000 to 4000 psig. Preferably, the carbon dioxide has a modifier or cosolvent, in the sense that the cosolvent is carried in the carbon dioxide in the nature of a dissolved constituent. A preferred cosolvent is an alcohol, such as methanol or ethanol.

Aspects of the present invention employ materials known as supercritical, critical or near-critical fluids. A material becomes a critical fluid at conditions which equal its critical temperature and critical pressure. A material becomes a supercritical fluid at conditions which equal or exceed both its critical temperature and critical pressure. The parameters of critical temperature and critical pressure are intrinsic thermodynamic properties of all sufficiently stable pure compounds and mixtures. Carbon dioxide, for example, becomes a supercritical fluid at conditions which equal or exceed its critical temperature of 31.1° C. and its critical pressure of 72.9 atm (1,070 psig). In the supercritical fluid region, normally gaseous substances such as carbon dioxide become dense phase fluids which have been observed to exhibit greatly enhanced solvating power. At a pressure of 3,000 psig (204 atm) and a temperature of 40° C., carbon dioxide has a density of approximately 0.845 g/cc and behaves much like a nonpolar organic solvent, having a dipole moment of zero Debyes.

A supercritical fluid displays a wide spectrum of solvation power as its density is strongly dependent upon temperature and pressure. Temperature changes of tens of degrees or pressure changes by tens of atmospheres can change a compound solubility in a supercritical fluid by an order of magnitude or more. This feature allows for the fine-tuning of solvation power and the fractionation of mixed solutes. The selectivity of nonpolar supercritical fluid solvents can also be enhanced by addition of compounds known as modifiers (also referred to as entrainers or cosolvents). These cosolvents are typically somewhat polar organic solvents such as acetone, ethanol, methanol, methylene chloride or ethyl acetate. Varying the proportion of cosolvent allows wide latitude in the variation of solvent power.

Supercritical, near critical and critical fluids can exhibit liquid-like density yet still retain gas-like properties of high diffusivity and low viscosity. The latter increases mass transfer rates, significantly reducing processing times. Additionally, the ultra-low surface tension of supercritical fluids allows facile penetration into microporous materials, increasing extraction efficiency and overall yields.

A material at conditions that border its supercritical state will have properties that are similar to those of the substance in the supercritical state. These so-called “near-critical” fluids are also useful for the practice of this invention. For the purposes of this invention, a near-critical fluid is defined as a fluid which is (a) at a temperature between its critical temperature (Tc) and 75% of its critical temperature and at a pressure at least 75% of its critical pressure, or (b) at a pressure between its critical pressure (Pc) and 75% of its critical pressure and at a temperature at least 75% of its critical temperature. In this definition, pressure and temperature are defined on absolute scales, e.g., Kelvin and psia. To simplify the terminology, materials which are utilized under conditions which are supercritical, near-critical or exactly at their critical point with or without polar cosolvent will jointly be referred to as “SCCNC” fluids or referred to as “SFS.”

SCCNC fluids can be used for the fractional extraction and manufacturing of highly purified gingerols and shogaols.

Embodiments of the present invention are directed to methods of using supercritical fluids for isolating and manufacturing gingerols for use as a therapeutic to treat nausea and emesis.

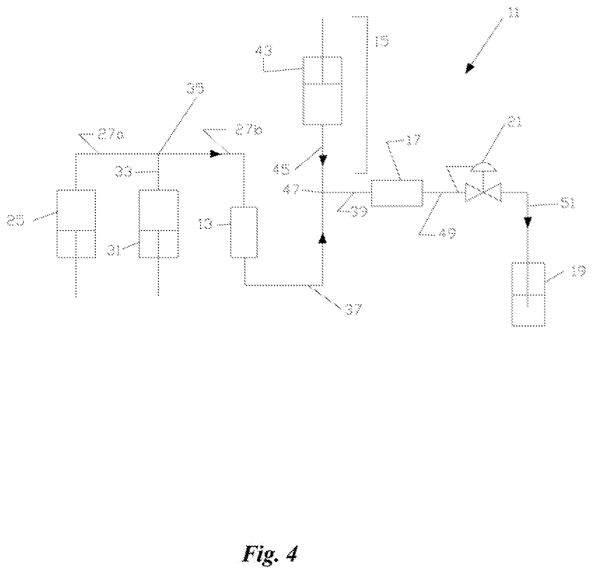

The present method and apparatus will be described with respect to FIG. 4 which depicts in schematic form the ginger fractionation apparatus, generally designated by the numeral 11.

Polarity-guided SCCNC fractionation can be carried out on the dried and fresh ginger powder. SCCNC CXF fractionations can be carried out on an automated extractor or a manual version of the same. As shown in FIG. 4, this is a dual pump system, utilizing syringe pump 25 for neat critical fluid (e.g. CO₂) and syringe pump 31 for cosolvent (e.g., ethanol).

After loading ginger into a cartridge on the cartridge holder 17, the fractionation procedure can start. For example, the system will be brought to 3,000 psig and 40° C., and extracted for 10 minutes with pure CO2. This fraction will be collected in ethanol in a glass vial, numbered 19 in FIG. 4. The extraction parameters will be then set to: Supercritical CO2 at 3,000 psig and extraction temperature 40° C., step extractions with ethanol as cosolvent at 5, 10, 20, 30 and 40 vol % each step being 10 min. Each biomass sample will yield 6 fractions, and which will be collected in ethanol in separate glass vials. The fractions will be dried under vacuum in a SpeedVac, and analyzed by HPLC for gingerols, zingerone, and shogaol content. Conditions which provide the highest combined content of gingerols and shogaol with ratios of 6-gingerol to 6-shogaol between 0.04 to 0.4 are favored for manufacturing larger quantities.

### EXAMPLES

### Example 1: Fractionation of Ginger Rhizome

Biomas: Zingiber officinale biomass, both fresh and dried, were obtained from reputable suppliers in Brazil. The material was shipped on ice by overnight freight to our facilities in Woburn, Mass. On receipt, the biomass samples were logged in; dried biomass was stored in dry, low humidity conditions and the fresh biomass was stored at 4° C. Samples were ground to a fine powder and extracted with different solvents—ethanol, methylene chloride, chloroform and hexane—to define the gingerol content of the material by HPLC analytical techniques. Samples of the underground biomass were used for cultivar identification and sent to outside contractors for heavy metals, herbicides and pesticides analyses. Small voucher samples were retained.

Ginger Powder: The dried ginger root was cut into chunks and dried in a convective oven at 37° C. for 24 hours to remove moisture. The biomass was then ground into a fine powder in a plate and hammer mill. A sample of this fine powder was also extracted by conventional techniques to re-establish the gingerols and shogaol content of the dried and ground Zingiber officinale biomass. The biomass powder was labeled and stored at −20° C.

The fresh ginger root was also cut into chunks and dried in a VirTis shelf freeze-dryer over a 24-hour period to remove all water and moisture. The biomass was then ground into a fine powder in a plate and hammer mill. A sample of this fine powder was also extracted by conventional techniques to re-establish the gingerols and shogaol content of the dried and ground Zingiber officinale biomass. The biomass powder was labeled and stored at −20° C.

Ginter Extract: Polarity-guided SCCNC fractionation was carried out on the dried and fresh ginger powder. As shown in FIG. 4, this is a dual pump system, utilizing syringe pump 25 for neat critical fluid (e.g. CO2) and syringe pump 31 for modifier (e.g. ethanol).

The data suggests that the following percentages of gingerol and shogaol were obtained. 6-shogaol and 6-gingerol define a ratio and the ratio of 6-shogaol to 6-gingerol is 0.04 to 0.40. Although the applicant does not wish to be bound to any theory, it is believed this ratio of 6-shogaol to 6-gingerol improves the efficacy of the extract for the treatment of nausea.

The ginger rhizome has a starting mass and the extract has a mass associated with one or more of the following 6-gingerol, 8-gingerol, 10-gingerol, and 6-shogaol. The ratio of 6-gingerol, 8-gingerol, 10-gingerol, and 6-shogaol total mass to starting mass is 20-40%.

The extracts are characterized as having 15-25% 6-gingerol, 1-5% 8-gingerol, 1-5% 10-gingerol and 1-5% 6-shogaol. The extracted gingerols and shogaols define a percentage of the total biomass ranging from about 10-15% to about 25-35%.

### Example 2: Tablet Formulation of Gingerol Compounds with Iron Supplements

This example will present a tablet formulation for an iron supplement and a gingerol compound.

Each tablet to contain:

Ferrous sulfate 325 mg

Gingerol Extract 4.0 to 14 mg

Olive oil (in an amount to solubilize gingerol extract)

Sterotex (in an amount to bind ferrous sulfate) approximately 2.00 mg

Gingerol extract is combined with olive oil to produce solution, slowly combined and mixed with ferrous sulfate powder and serotex to form a compressible solid mass and the compressible solid mass is pressed into tablet form.

### Example 3: Gel Cap Formulation of Gingerol Compounds with Iron Supplements

Each gel cap to contain:

Ferrous Sulfate 325 mg

Gingerol Extract 4.0 to 14 mg

Olive oil (in an amount to solubilize gingerol extract)

Tocopherols (as a preservative)

Lecithin

Medium chain triglycerides

Gingerol extract, desired tocopherols, lecithin and medium chain triglycerides are solubilized in olive oil to form a gingerol-olive oil product. Ferrous sulfate, as a fine powder, is dispersed within the gingerol-olive oil product to form a ferrous sulfate suspension. The ferrous sulfate suspension is loaded into a gel cap as known in the art.

### Example 4: Solution of Gingerol Compounds and Iron Supplements

Each liquid dose (five milliliters) of solution to contain:

Ferrous Sulfate 325 mg

Gingerol Extract 4.0 to 14 mg

Olive oil (in an amount to solubilize gingerol extract)

Tocopherols (as a preservative)

Lecithin

Medium chain triglycerides

Gingerol extract, desired tocopherols, lecithin and medium chain triglycerides are solubilized in olive oil to form a gingerol-olive oil product. Ferrous sulfate, as a fine powder, is dispersed within the gingerol-olive oil product to form a ferrous sulfate suspension. Ferrous sulfate suspension is placed in suitable liquid dispensing vessel, with instructions to shake well.

### Example 5: Capsule Formulation of Gingerol Compounds with Iron Supplements

This example will present a capsule formulation for an iron supplement and a gingerol compound.

Ferrous sulfate 325 mg

Gingerol Extract 4.0 to 14 mg

Olive oil (in an amount to solubilize gingerol extract)

Sterotex (in an amount to bind ferrous sulfate) approximately 2.00 mg

Gingerol extract is combined with olive oil to produce solution, slowly combined and mixed with ferrous sulfate powder and Sterotex to form a powder mass and the powder mass is loaded into an appropriately sized capsule in a manner known in the art.

In each of the above example directed to a dosage form, ferrous sulfate, in whole or in part, can be readily substituted with an equivalent amount of 40-80 mg elemental iron with ferrous fumarate and ferrous gluconate and mixtures thereof.

The dosage forms are subject to many alterations and modification for special needs. Pediatric dosage forms may favor liquids to be administered in lower dosages of iron by dropper or the like. The oil base containing the gingerol composition or the gingerol composition without an oil base may be emulsified and the emulsion held in an aqueous medium.

Particularly for dosage forms intended to be administered as liquid, it is useful to use flavoring agents to improve patient acceptance.

Thus, the present invention has been described in detail in which a dosage form has a gingerol composition addressing the nausea and gastric distress of iron supplements and synergistically promotes hematopoiesis to treat anemia. The description is directed to the best mode presently contemplated and the inventor's opinion as to such best mode may change in time and embodiments of the present invention are subject to alteration and modification. Therefore, this description should not be considered limiting and the invention should comprise the subject matter of the claims which follow and their equivalents.

It is intended that the matter contained in the preceding description be interpreted in an illustrative rather than a limiting sense.

What is claimed is:

1. A dosage form for the treatment of iron deficiency anemia comprising an effective amount of an iron supplement selected from the group comprising ferrous sulfate, ferrous gluconate, ferrous fumarate and mixtures thereof with an effective amount of a gingerol composition to suppress nausea and/or gastric distress, said gingerol composition selected from the group of gingerol compounds comprising 6-gingerol, 8-gingerol, 10-gingerol, 12-gingerol, 6-shogaol, 8-shogaol and 10-shogaol as single compositions and mixtures thereof.

2. The dosage form of claim 1 further comprising an oil base wherein the iron supplement is dispersed as a solid suspension in said oil base and said gingerol composition dissolved in said oil base.

3. The dosage form of claim 2 wherein said oil base is held in a gel cap.

4. The dosage form of claim 1 wherein said gingerol composition is an extract from a ginger compound containing natural source.

5. The dosage form of claim 1 wherein said gingerol composition is used to wet a powder of said iron supplement to form a gingerol wetted iron supplement powder.

6. The dosage form of claim 5 wherein said gingerol wetted iron supplement powder is pressed into a tablet.

7. The dosage form of claim 5 wherein said gingerol wetted iron supplement powder is loaded into a capsule.

8. The dosage form of claim 1 wherein said iron supplement and gingerol are dissolved or suspended in a solution.

9. The dosage form of claim 1 wherein said iron supplement is ferrous sulfate.

10. The dosage form of claim 1 wherein said iron supplement is present in 40 to 80 mg of elemental iron.

11. The dosage form of claim 1 wherein said gingerol composition is present in an amount effective to promote hematopoiesis.

12. A method of treating anemia comprising the step of administering a dosage form comprising an effective amount of an iron supplement selected from the group comprising ferrous sulfate, ferrous gluconate, ferrous fumarate and mixtures thereof with an effective amount of a gingerol composition to suppress nausea and/or gastric distress, said gingerol composition selected from the group of gingerol compounds comprising 6-gingerol, 8-gingerol, 10-gingerol, 12-gingerol, 6-shogaol, 8-shogaol and 10-shogaol as single compositions and mixtures thereof.

13. The method of claim 12 wherein said dosage form has an oil base wherein the iron supplement is dispersed as a solid suspension in said oil base and said gingerol composition dissolved in said oil base.

14. The method of claim 13 wherein said oil base is held in a gel cap.

15. The method of claim 12 wherein said gingerol composition is an extract from a ginger compound containing natural source.

16. The method of claim 12 wherein said gingerol composition is used to wet a powder of said iron supplement to form a gingerol wetted iron supplement powder.

17. The method of claim 16 wherein said gingerol wetted iron supplement is pressed into a tablet.

18. The method of claim 16 wherein said gingerol wetted iron supplement is loaded into a capsule.

19. The method of claim 13 wherein said iron supplement and gingerol are dissolved or suspended in a solution.

20. The method of claim 12 wherein said iron supplement is present in 40 to 80 mg of elemental iron.
